(19) United States
(12) Patent Application Publication
(10) Pub. No.: US 2026/0060949 A1
(43) Pub. Date: Mar. 5, 2026

(54) METHOD FOR TREATING DAMAGE INDUCED BY SLEEP DEPRIVATION

(71) Applicant: President and Fellows of Harvard College, Cambridge, MA (US)

(72) Inventors: Dragana ROGULJA, Cambridge, MA (US); Alen JUGINOVIC, Cambridge, MA (US)

(73) Assignee: President and Fellows of Harvard College, Cambridge, MA (US)

(21) Appl. No.: 19/314,259

(22) Filed: Aug. 29, 2025

Related U.S. Application Data
(60) Provisional application No. 63/688,419, filed on Aug. 29, 2024.

(51) Int. Cl. A61K 31/192; A61K 31/575; A61K 45/06; A61P 3/02; A61P 25/00

(52) U.S. Cl. CPC A61K 31/192; A61K 31/575; A61K 45/06; A61P 3/02; A61P 25/00

(57) ABSTRACT

Described herein are methods and compositions for the use of treating damage induced by SD. Aspects of the invention relate to administering to a subject in need thereof an agent that reduces ER stress levels. In some embodiments, administration of an agent that reduces ER stress levels repairs SD-induced damage in the gut.

### CROSS-REFERENCE TO RELATED APPLICATION

This Application claims benefit under 35 U.S.C. § 119(e) of the U.S. Provisional Application No. 63/688,419 filed Aug. 29, 2024, the contents of which are incorporated herein by reference in their entirety.

### FIELD OF THE INVENTION

The field of the invention relates to methods for the treatment or prevention of sleep deprivation-induced damage.

### BACKGROUND

Sleep is an essential, widespread behavior which becomes fragmented and shortened with age. This negatively affects cognitive functions like learning, remembering and decision-making, and is a major risk factor for multiple diseases. Poor sleep is not limited to the elderly though—most of us experience significant sleep problems at least occasionally. Research described herein addresses changes that occur in the body after sleep deprivation, and demonstrates ways to counter those changes to offset or ameliorate its negative effect.

### SUMMARY

The methods and compositions disclosed herein are based, in part, on the discovery that sleep deprivation (SD) results in increased endoplasmic reticulum (ER) stress associated with nutrient malabsorption. Accordingly, one aspect provided herein describes a method for treating damage induced by sleep deprivation (SD), the method comprising administering to an individual who is sleep deprived an agent that reduces ER stress.

In one embodiment of any aspect provided herein, the damage is nutrient malabsorption. In one embodiment of any aspect provided herein, the damage occurs at a site selected from the group consisting of: brain, gastrointestinal tract, mouth, throat, lungs, heart, liver, gut, stomach, kidney, skin, bones, large intestine, small intestine, bladder, and muscular system.

In one embodiment of any aspect provided herein, the damage occurs in the gut.

In one embodiment of any aspect provided herein, SD is chronic or acute.

In one embodiment of any aspect provided herein, the agent is selected from the group consisting of: a compound, a small molecule, a food additive, and an enzyme. In one embodiment of any aspect provided herein, the agent is a small molecule.

Exemplary small molecule inhibitors of ER stress are presented herein in FIGS. 6A-6I. In one embodiment of any aspect provided herein, the small molecule is 4-Phenylbutyric acid or Tauroursodeoxycholic acid.

In one embodiment of any aspect provided herein, the method further comprises the step of, prior to administering, diagnosing the individual of having or being at risk of having damage induced by SD.

In one embodiment of any aspect provided herein, the method further comprises the step of, prior to administering, receiving a diagnosis or results of an assay diagnosing the individual as having or being at risk of having damage induced by SD.

Another aspect disclosed herein provides a composition for treating or preventing damage induced by SD, the composition comprising an agent that reduces ER stress and a sedative.

Another aspect disclosed herein provides a composition for treating or preventing damage induced by SD, the composition comprising: an agent that reduces ER stress and a stimulant.

In one embodiment of any aspect provided herein, the agent is a small molecule selected from 4-Phenylbutyric acid or Tauroursodeoxycholic acid.

In one embodiment of any aspect provided herein, the sedative is selected from a group consisting of: a barbiturate, a benzodiazepine, a non-benzodiazepine hypnotic, a methoaqualone, a first generation antihistamine, an antidepressant, an antipsychotic, an herbal sedative, alcohol, an opioid, a general anesthetic, a melatonin agonist, an orexin antagonist, and a skeletal muscle relaxant.

In one embodiment of any aspect provided herein, the stimulant is selected from a group consisting of: an herbal stimulant, an amphetamine, a methamphetamine, cocaine, a methylxanthine, ephedrine, a cathinone, mephedrone, methylenedioxypyrovalerone, methylenedioxymethamphetamine, nicotine, propylhexedrine, and pseudoephedrine.

In one embodiment of any aspect provided herein, the composition further comprises a pharmaceutically acceptable carrier.

Another aspect disclosed herein provides a method for treating or preventing damage induced by SD, the method comprising: administering to an individual who is sleep deprived any of the compositions disclosed herein.

### Definitions

As used herein, the term “sleep deprivation” refers to an individual getting at least 10% less sleep than recommended (e.g., by the National Sleep Foundation) for their age group, and includes at least 20%, or at least 30%, or at least 40%, or at least 50%, or at least 60%, or at least 70%, or at least 80%, or at least 90% or even a greater percentage less sleep than recommended for their age group.

The terms “lower”, “reduced”, “reduction” or “decrease”, “down-regulate” or “inhibit” are all used herein generally to mean a decrease by a statistically significant amount. However, for avoidance of doubt, “lower”, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level. When “decrease”, “reduction”, or “inhibition” is used in the context of the ER stress levels and/or damage, it refers to a reduction in ER stress levels and/or damage in a cell, a tissue, a cell extract, or a cell supernatant.

The terms “increased”, “increase”, or “activate” are all used herein to mean an increase by a statically significant amount. In some embodiments, the terms “increased”, “increase”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

The terms “significantly different than,” “statistically significant,” and similar phrases refer to comparisons between data or other measurements, wherein the differences between two compared individuals or groups are evidently or reasonably different to the trained observer, or statistically significant (if the phrase includes the term “statistically” or if there is some indication of statistical test, such as a p-value, or if the data, when analyzed, produce a statistical difference by standard statistical tests known in the art).

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder. The term “treating” includes reducing or alleviating at least one adverse effect or symptom of a condition, disease or disorder, for example ER stress damage induced by SD. Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of a disease or disorder, diminishment of extent of a disease or disorder, stabilized (i.e., not worsening) state of a disease or disorder, delay or slowing of progression of a disease or disorder, amelioration or palliation of the disease or disorder state, and remission (whether partial or total), whether detectable or undetectable. The term “treatment” of a disease or disorder also includes providing relief from the symptoms or side-effects of the disease (including palliative treatment).

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material that maintains a drug or other agent in a form for delivery to a subject. Each carrier must be “acceptable” in the sense of being compatible with the other ingredients of the formulation, for example the carrier does not decrease the impact of the agent on the treatment. In other words, a carrier is pharmaceutically inert. The terms “physiologically tolerable carriers” and “biocompatible delivery vehicles” are used interchangeably.

The terms “administered” and “subjected” are used interchangeably in the context of treatment of a disease or disorder. Both terms refer to a subject being treated with an effective dose of pharmaceutical composition comprising a composition as described herein by methods of administration such as parenteral or systemic administration.

The term “optional” or “optionally” means that the subsequent described event, circumstance or substituent may or may not occur, and that the description includes instances where the event or circumstance occurs and instances where it does not.

As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of elements that do not materially affect the basic and novel or functional characteristic(s) of that embodiment of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides exemplary micrographs of lipids in control and SD mice. Confocal images of the mouse small intestine fluorescently labeled with BODIPY (green, which binds to neutral lipids, such as those from diet). After 5 days of sleep deprivation, lipid levels are elevated in the intestinal epithelium (right) compared to controls (no sleep restriction). In the control, some green is seen inside the cells—the lipids which are absorbed are passed into the circulation. In sleep-deprived mice, the lipids are stuck inside the cells. The image shows a section of ˜1000×1000 micrometers (1 mm²) of the small intestine.

FIG. 2 provides exemplary electron microscopy (EM) images of the mouse small intestine showing that lipids accumulate inside the enterocytes of sleep-restricted mice. Scale bars below the images, 2 μm. Red arrows point to lipids, which accumulate inside and around enterocytes (absorptive cells of the gut) in sleep-deprived mice (5 days of sleep deprivation). Blue arrows point to large lipoprotein particles (aggregates) which form inside the endoplasmic reticulum, discussed below and shown closer in FIG. 4.

FIG. 3 provides exemplary data showing that levels of triglycerides in the serum of sleep-deprived mice are lower than in the controls. Each dot represents one mouse.

FIG. 4 provides exemplary data showing EM images from mice that were sleep-deprived for 1 day. The scale bar below the image is 100 nm. Red arrows point to large lipoprotein particles which are inside the ER. Green arrows point to ribosomes on the ER surface.

FIG. 5 provides exemplary data showing that several proteins which are required for lipid processing are depleted after one day of sleep deprivation (1 SD). (Bottom) ER stress markers are elevated after 1 day of sleep deprivation. 5 SD=5 days of sleep deprivation. NON SD—non-deprived controls.

FIGS. 6A-6I provide the chemical structures of (FIG. 6A) sodium phenylbutyrate, (FIG. 6B) salubrinal, (FIG. 6C) Integrated Stress Response inhibitor (ISRIB), (FIG. 6D) GSK2606414, (FIG. 6E) GSK2656157, (FIG. 6F) 4μ8C, (FIG. 6G) STF-083010, (FIG. 6H) MKC-8866, and (FIG. 6I) KIRA6.

FIGS. 7A-7B provide an illustration (FIG. 7A) and exemplary EM images (FIG. 7B) of the tight junctions between enterocytes in control or SD mice. Scale bar below the EM images is 200 nm. The tight junctions between enterocytes are disrupted in sleep-deprived mice (right, 5 days of sleep deprivation). The spaces between cells are filled with lipids, and with particles which appear to be chylomicrons.

### DETAILED DESCRIPTION

Sleep deprivation or sleep disruption can occur as a symptom of a number of disorders, as well as from the pressures and demands of life in present times. Apart from medical disorders, certain occupations and life circumstances can lead to chronic or acute SD and to the negative consequences thereof. For example, soldiers, doctors, students, travelers and parents of newborns or babies, jet-lagged travelers, among others, frequently experience at least acute SD. As described herein, the present invention is based in part on the discovery that SD increases ER stress in the intestinal gut, resulting in damage, e.g., nutrient malabsorption. Nutrient malabsorption refers to a reduced capacity of the gastrointestinal tract to absorb macronutrients (carbohydrates, proteins, fats) and/or micronutrients (vitamins, minerals) from ingested food. Data provided herein show that increased levels of ER stress induced by SD leads to nutrient malabsorption. Malabsorption may be caused by primary disorders of the intestine, pancreas, or liver, or may occur secondary to infections, inflammatory diseases, surgical resections, or genetic abnormalities affecting nutrient processing. However, malabsorption associated with SD can occur in subjects with or without these primary or secondary conditions.

Damage was ameliorated following the administration of an agent that inhibits ER stress. Thus, reducing ER stress in a sleep deprived individual reversed damage associated with SD and can provide an effective treatment for at least some of the negative effects of SD. The following discusses the methods, compositions, and consideration necessary to practice the technology described herein.

### Sleep Deprivation (SD)

SD generally occurs when an individual gets less sleep than needed to feel awake and alert. SD includes the absence of sleep in at least a consecutive 24 hour period, as well as a reduced amount of sleep (e.g., reduced by at least 20%, but including, for example, reduced by at least 30%, at least 40%, at least 50%, at least 60%, at least 70%, at least 80%, at least 90% or more) in at least a consecutive 24 hour period relative to that recommended for an individual in a given age and health status group. Individuals vary in the amount of sleep they require, largely depending upon age. Some, such as older adults, tend to be more resistant to the effects of sleep deprivation, while others, especially children and young adults, are more vulnerable. The National Sleep Foundation (NSF) recommendations for appropriate sleep durations for specific age groups are: newborns (0-3 months): 14-17 hours each day, infants (4-11 months): 12-15 hours, toddlers (1-2 years): 11-14 hours, preschoolers (3-5): 10-13 hours, school-age children (6-13): 9-11 hours, teenagers (14-17): 8-10 hours, adults (18-64): 7-9 hours, older adults (65+): 7-8 hours.

In some embodiments, SD is acute. Acute sleep deprivation refers to a period of seven or fewer, e.g., six or fewer, five or fewer, four or fewer, three or fewer, or two or fewer consecutive 24 hour days in which an individual gets at least 20% less sleep than is recommended for an individual of their age and health status. Acute SD is frequently, but not always, associated with certain occupations or circumstances, e.g., physicians in training or on call, emergency responders or disaster workers, soldiers, travelers, and students studying for exams. Jetlag generally involves acute SD, or at least acute disturbance of the normal sleep pattern, and is contemplated as a condition that can benefit from the compositions and methods described herein.

In some embodiments, SD is chronic. Chronic sleep deprivation refers to a prolonged period of time (weeks, months, or years) without sufficient sleep or with reduced sleep (e.g., reduced by at least 10%) relative to that recommended for a subject's age group. Chronic sleep deprivation is frequently, but not necessarily, associated with medical conditions or anxiety that perturb the normal sleep pattern.

In the short term, a lack of adequate sleep can affect judgment, mood, ability to learn and retain information, and may increase the risk of serious accidents and injury. In the long term, chronic sleep deprivation may lead to a host of health problems including obesity, diabetes, cardiovascular disease, and even early mortality. SD may result in, or increase the risk for, depression, loss of memory, hallucinations, psychosis, increased blood pressure, increased stress hormone levels, seizures, headaches, weight gain or weight loss, increased risk of diabetes, and an increased risk of fibromyalgia.

Extensive tissue damage has been found in sleep deprived individuals in various organs in animal models. Cell death, cell death signaling, and cellular damage have been observed following SD. After several nights of SD, it has been observed that locus coeruleus (LC) brain cells died in a mouse model. In the liver, a strong increase in cellular damage, including DNA damage, was observed in a SD rat. In the lung and spleen of the SD rat, an increase in cell death signaling and cellular damage was observed. In the intestine, cellular damage, including DNA damage, and cell death have been observed following SD.

In various embodiments of the aspects described herein, SD-induced cellular damage and/or death occurs in various organs and/or sites within the body. Non-limiting examples of sites where SD-induced damage can occur include brain, gastrointestinal tract, mouth, throat, lungs, heart, liver, gut, stomach, kidney, skin, bones, large intestine, small intestine, bladder, and muscular system.

Agents that Reduce ER Stress

Agents that reduce ER stress as described herein or as known in the art can be administered in amounts and formulations as described herein or as known in the art to exert effects on levels or activities of ER stress. The ER is a membrane-bound organelle responsible for the folding, maturation, and trafficking of secretory and membrane proteins, as well as for maintaining intracellular calcium and lipid homeostasis. Various physiological and pathological stimuli, e.g., such as hypoxia, nutrient malabsorption, oxidative stress, mutations in client proteins, or disturbances in calcium balance, can disrupt ER function and lead to the accumulation of misfolded or unfolded proteins within the ER lumen. This condition is referred to as ER stress.

In response to ER stress, cells activate a highly conserved signaling network known as the unfolded protein response (UPR), mediated primarily by the sensor proteins PERK, IRE1, and ATF6. The UPR aims to restore homeostasis by reducing global protein translation, increasing the production of molecular chaperones, and enhancing degradation of misfolded proteins. When ER stress is prolonged or severe, however, UPR signaling can shift from pro-survival to pro-apoptotic pathways, contributing to the pathogenesis of a wide range of disorders, including neurodegenerative diseases, diabetes, cardiovascular disease, cancer, and inflammatory conditions.

As used herein, the term “ER stress inhibitor” refers to any agent that decreases the level or activity of one or more markers of ER stress or the unfolded protein response (UPR) in a cell, tissue, or organism. ER stress levels can be measured, for example, by changes in the expression or activation state of ER stress-associated proteins (e.g., BiP/GRP78, CHOP/DDIT3, ATF4, phosphorylated eIF2α), splicing of XBP1 mRNA, activation of ATF6, or other molecular or phenotypic indicators as described herein.

A variety of structurally and mechanistically distinct classes of ER stress inhibitors have been described. Without limitation, such classes include the following:

Chemical Chaperones and Proteostasis Enhancers—These compounds facilitate proper protein folding within the ER lumen, thereby reducing the accumulation of misfolded proteins that trigger the UPR. Exemplary chemical chaperones include 4-phenylbutyric acid (4-PBA), sodium phenylbutyrate, and tauroursodeoxycholic acid (TUDCA). These agents act in a generally broad fashion, enhancing ER folding capacity and alleviating proteotoxic stress.

Modulators of the eIF2α Integrated Stress Response (ISR) Branch—This class targets signaling downstream of PERK-mediated phosphorylation of eIF2α, which controls translation attenuation during ER stress. For example, salubrinal inhibits eIF2α dephosphorylation, maintaining p-eIF2α levels and reducing ER stress-associated apoptosis. Integrated Stress Response Inhibitor (ISRIB) acts at the level of eIF2B to restore translation despite persistent p-eIF2α, thereby modulating stress signaling output.

PERKPathway Inhibitors—These small molecules inhibit the kinase activity of PERK (protein kinase RNA-like ER kinase), a proximal UPR sensor. Representative examples include GSK2606414 and GSK2656157, which reduce PERK autophosphorylation and downstream eIF2α phosphorylation. Such inhibitors may be useful in settings where chronic PERK activation is deleterious.

IRE1 RNase Kinase Inhibitors—IRE1α is a dual kinase/RNase UPR sensor that mediates unconventional splicing of XBP1 mRNA. Inhibitors of IRE1 RNase activity reduce XBP1s production and downstream transcriptional programs. Examples include 4 8C, STF-083010, MKC-8866, and members of the KIRA series (e.g., KIRA6), which act allosterically via the kinase domain. These agents may also attenuate regulated IRE1-dependent mRNA decay (RIDD).

ATF6 Pathway Modulators ATF6 is a membrane-bound transcription factor activated during ER stress via regulated intramembrane proteolysis. While fewer selective inhibitors exist, modulation of ATF6 signaling can be achieved indirectly by chemical chaperones or experimental small molecules identified in phenotypic screens.

Additional compounds, including certain natural products, experimental kinase inhibitors, and biologics (e.g., antibodies against stress pathway components), have been reported to reduce ER stress or UPR activation. These may act through mixed or indirect mechanisms, such as modulation of ER calcium homeostasis, redox state, or protein trafficking.

In certain embodiments, the ER stress inhibitor may be any compound falling within one or more of the foregoing classes, a pharmaceutically acceptable salt, solvate, prodrug, analog, or derivative thereof, or a combination thereof.

In one embodiment, the agent that inhibits ER stress is 4-Phenylbutyric acid or Tauroursodeoxycholic acid.

4-Phenylbutyric acid, also known 4-PBA, is a selective inhibitor of ER stress having a chemical

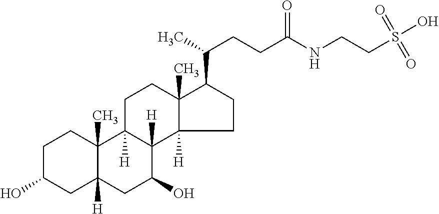

structure as shown below.

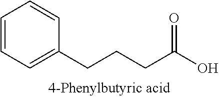 [SMILES: O=C(O)CCCc1ccccc1]

Tauroursodeoxycholic acid, also known as ursodoxicoltaurine, is a highly hydrophilic tertiary bile acid having a chemical structure as shown below.

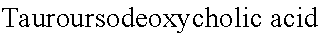

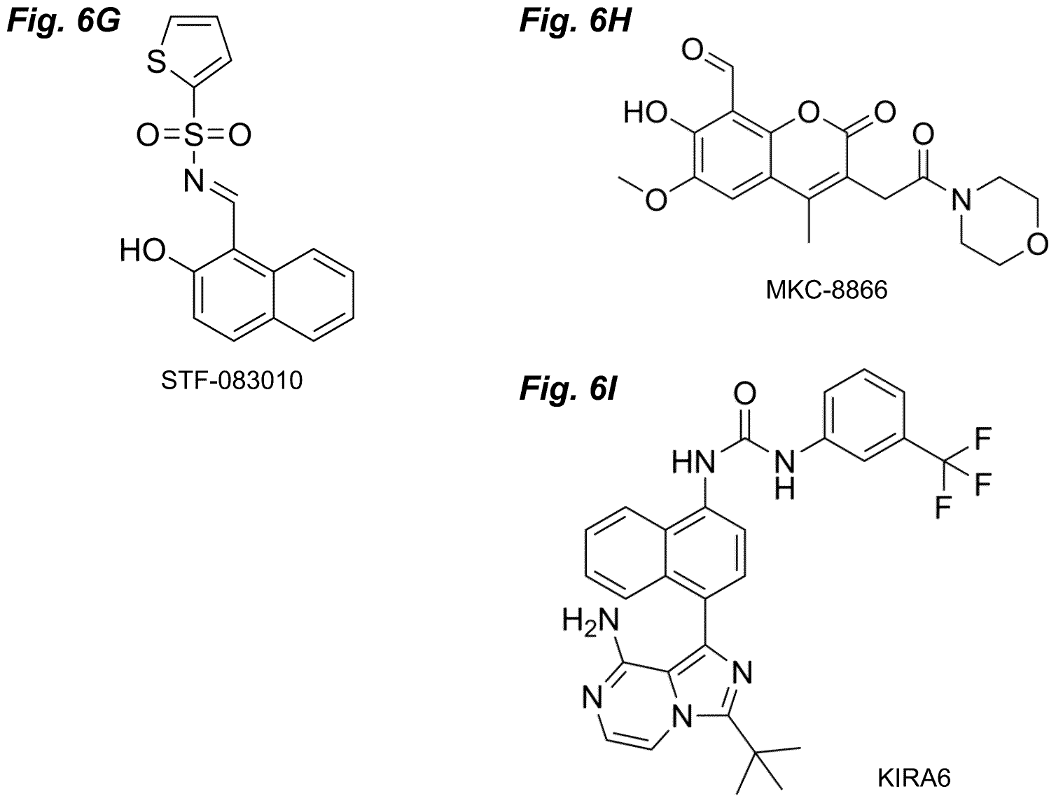

In one embodiment, the agent that inhibits ER stress is selected from the list consisting of sodium phenylbutyrate, salubrinal, ISRIB, GSK2606414, GSK2656157, 4μ8C, STF-083010, MKC-8866, and KIRA6. Structures for sodium phenylbutyrate, salubrinal, ISRIB, GSK2606414, GSK2656157, 4μ8C, STF-083010, MKC-8866, and KIRA6 are disclosed herein, e.g., in FIGS. 6A-6I, respectively.

### Therapeutic Compositions and Methods

In various embodiments, the damaging effects of SD, and in particular embodiments, the damaging effects of SD on the gut, can be prevented, treated or ameliorated by administration of an agent that reduces ER stress levels as described herein. Therapeutic methods, as well as formulations are discussed in the following.

In one embodiment, a composition for treating the damaging effects of SD includes an agent that reduces ER stress levels as described herein or as known in the art. In one aspect, a composition comprising an agent that reduces ER stress levels is administered to an individual who is, or is likely to become, sleep-deprived, to treat and/or prevent SD-induced tissue damage, e.g., nutrient malabsorption. In one embodiment, the increased ER stress level occurs in the intestinal gut. In one embodiment, the increased ER stress level-induced damage occurs in the intestinal gut.

Agents that reduce ER stress levels as described herein or as known in the art can be administered in amounts and formulations as described herein or as known in the art to exert effects on levels or activities of ER stress.

In one aspect, a composition for treating the damaging effects of SD includes an agent that reduces ER stress levels as described herein or as known in the art and a sedative. In one aspect, a composition comprising an agent that reduces ER stress levels and a sedative is administered to an individual who is sleep-deprived to treat and/or prevent increased ER stress levels and tissue damage that occurs with SD. In one embodiment of these aspects, the administration of a composition comprising an agent that reduces ER stress levels is administered to an individual who is likely to become sleep-deprived, so as to ameliorate and/or prevent an SD-induced increase of ER stress level and/or issue damage. In another embodiment of these aspects, the administration prevents or treats the increase of ER stress levels and/or tissue damage in the intestinal gut. In one embodiment, the method of treatment or prevention further comprises the step, before the administering step, of selecting an individual who is or is at risk of becoming sleep-deprived.

A “sedative” is a substance that induces sedation by reducing irritability or excitement. Sedatives can promote or induce sleep. It is contemplated that administration of a sedative, in conjunction with an agent that reduces ER stress levels as described herein can both assist sleep in an individual who is unable to fall asleep or to remain asleep, and counter or ameliorate the increase of ER stress levels and/or tissue damage that normally occurs with SD. It is contemplated that administering a sedative, in conjunction with an agent that reduces ER stress levels as described herein can also promote more rapid recovery from SD than would occur without such administration. Doses of sedatives such as benzodiazepines, when used as a hypnotic to induce sleep, tend to be higher than amounts used to relieve anxiety, whereas only low doses are needed to provide a peaceful effect. As used herein, the term “sedation” refers to calm, relaxation, or sleep due to the intake of a sedative.

Non-limiting examples of sedatives include barbiturates, benzodiazepines, non-benzodiazepine hypnotics, methoaqualones, first generation antihistamines, antidepressants, antipsychotics, herbal sedatives, alcohol, opioids, general anesthetics, melatonin agonists, orexin antagonists, and skeletal muscle relaxants. For purposes of compositions including a sedative, it should be understood that the sedative is present and administered in an amount sufficient to have a sedative effect. For example, while alcohol (ethanol) can have a sedative effect, it is also used as a solvent for some pharmaceutical formulations; however, in these instances, ethanol is generally not present in an amount sufficient to have a significant sedative effect.

In one aspect, a composition for treating the damaging effects of SD comprises an agent that reduces ER stress levels as described herein or as known in the art, and a stimulant. In another aspect, a composition comprising an agent that reduces ER stress levels and a stimulant is administered to an individual who is sleep-deprived to treat and/or prevent increased ER stress levels and/or tissue damage. In one embodiment of these aspects, the administration of a composition comprising an agent that reduces ER stress levels and a stimulant is to an individual who is likely to become sleep-deprived, so as to ameliorate and/or prevent SD-induced increase of ER stress levels and/or tissue damage. In another embodiment of these aspects, the administration prevents or treats the increase of ER stress levels and/or tissue damage in the intestinal gut. In another embodiment, the method of treatment or prevention further comprises the step, before the administering step, of selecting an individual who is or is at risk of becoming sleep-deprived.

“Stimulant” is an overarching term that encompasses drugs that increase alertness or activity of the body, drugs that are invigorating, and/or drugs that have sympathomimetic effects. In general, a stimulant can keep an individual awake longer than they would remain awake without the stimulant. It is contemplated that administration of a stimulant, in conjunction with an agent that reduces ER stress levels as described herein can both keep awake an individual who needs to stay awake, and prevent, or at least ameliorate the increase of ER stress levels and/or tissue damage that normally occurs with SD. Stimulants are widely used throughout the world as prescription medicines as well as without a prescription (either legally or illicitly) as performance-enhancing or recreational drugs.

Non-limiting examples of stimulants include herbal stimulants, amphetamines, methamphetamines, cocaine, methylxanthines, ephedrine, cathinones, mephedrone, methylenedioxypyrovalerone, methylenedioxymethamphetamine, nicotine, propylhexedrine, and pseudoephedrine.

Various embodiments noted herein involve selecting an individual who is sleep-deprived, diagnosing someone as being sleep-deprived, receive the results of an assay that diagnoses a person as being sleep-deprived, or administering an agent that reduces ER stress levels to an individual who is sleep deprived. A sleep deprived individual is defined as one who is getting at least 10%, or at least 20%, or at least 30%, or at least 40%, or at least 50%, or at least 60%, or at least 70%, or at least 80%, or at least 90% or more, less sleep than recommended for their age group.

As used herein, the term “reduce” when used in reference to the level or activity of a targeted product, e.g., ER stress levels, refers to a decrease in the level and/or activity of the targeted product by at least 10% or more, e.g. by 10% or more, 50% or more, 70% or more, 80% or more, 90% or more, 95% or more, or 98% or more as compared to an appropriate control.

As used herein, an appropriate control can refer to the ER stress levels in a subject prior to receiving the agent that inhibits ER stress, or an otherwise similar subject that does not receive the agent that inhibits ER stress as disclosed herein. The efficacy of an inhibitor of a particular target, e.g. its ability to decrease the level and/or activity of the target (e.g., ER stress levels) can be determined, e.g. by measuring the level of an expression product and/or the activity of the target. Any appropriate method can be used; however, one way to measure ER stress levels is to use Western blot analysis, immunoassay, or qPCR analysis to quantify the protein or mRNA levels of relevant markers. The following are examples of markers that are useful to assess ER stress levels: for blood or circulating markers, XBP1s or spliced XBP1 mRNA, GRP78/BiP, and CHOP (from certain types of white blood cells). For stool, exploratory studies suggest that GRP78/BiP can be detectable in the stool as a potential non-invasive marker of intestinal ER stress. Additional methods are known in the art and/or described in the Examples herein.

Following protein synthesis, secretory, intra-organellar, and transmembrane proteins translocate into the endoplasmic reticulum (ER) where they are post-translationally modified and properly folded. The accumulation of unfolded proteins within the ER triggers an adaptive mechanism known as the unfolded protein response (UPR) that counteracts compromised protein folding. The transmembrane serine/threonine kinase IRE1, initially identified in Saccharomyces cerevisiae, is a proximal sensor for the UPR that transmits the unfolded protein signal across the ER membrane. The human homolog IRE1α was later identified and is ubiquitously expressed in human tissues. Upon activation of the unfolded protein response, IRE1α splices X-box binding protein 1 (XBP-1) mRNA through an unconventional mechanism using its endoribonuclease activity. This reaction results in expression of a splicing variant isoform of XBP-1, XBP-1s, which is a potent transcriptional activator that induces expression of many unfolded protein response (UPR) responsive genes. XBP1s is not typically directly measured in blood; instead, its presence and activity are inferred from the levels of spliced mRNA encoding XBP1s or the expression of its downstream target genes in blood cells. Assays that detect XBP1 splicing, such as RT-PCR are used on RNA from cells isolated from blood samples, like peripheral blood mononuclear cells (PBMCs. PCR assays for XBP-1 splicing are known in the art and described, for example, by Yoshida et al., Cell 107: 881-891 (2001). Gene expression profiling can identify the signature of XBP1s activation. Western blot (WB) can also be used to directly detect the XBP1s protein, after isolating specific cells from a blood sample (e.g., lymphocytes, AML cells from bone marrow or peripheral blood). Alternatively, Flow Cytometry can be used to detect XBP1s protein levels within specific cell populations after they have been isolated and stained with antibodies. Antibodies specific for the XBP-1s isoform are available from Cell Signaling Technology (CST), Cat #40435.

To help proteins fold properly, the ER contains a pool of molecular chaperones including BiP. BiP was identified as an immunoglobulin heavy chain binding protein in pre-B cells. It was also found to be induced at the protein level by glucose starvation. When protein folding is disturbed inside ER, BiP synthesis is increased. Subsequently, BiP binds to misfolded proteins, preventing them from forming aggregates and assisting in their proper refolding. GRP78/BiP is primarily an intracellular endoplasmic reticulum (ER) chaperone, though it can be found on the cell surface, particularly on tumor cells, and is shed into the extracellular environment. GRP78 can be detected, e.g., in blood, serum, stool or in other samples by Western Blot, ELISA or mass spectrometry. Anti-GRP78/Bip antibody is available from Cell Signaling Technology (CST), Cat #3177. Commercially available ELISA kits for the detection of GRP78 are available; see e.g., Enzo life Sciences Cat #ADI-900-214 and Abcam Cat #ab302765. The baseline for GRP78/BiP in serum is generally between 3-6 μg/mL. Higher levels indicate ER stress. For example, ranges of GRP78/BiP serum concentrations were higher in patients with obesity, diabetes mellitus, and metabolic syndrome (which are examples of clinical scenario for ER stress) compared with patients without metabolic disturbances (5.67 (3.74-11.62) μg/mL vs. 9.15 (5.74-16.38) μg/mL, p<0.001; 4.72 (3.63-9.94) μg/mL vs. 8.57 (5.27-16.73) μg/mL, p<0.001 and 4.15 (2.91-5.76) μg/mL vs. 9.15 (5.85-16.73) μg/mL, p<0.001, respectively).

CHOP was identified as a C/EBP-homologous protein that inhibits C/EBP and LAP in a dominant-negative manner. CHOP expression is induced by specific cellular stresses, and the induced CHOP suppresses cell cycle progression from G1 to S phase. During ER stress, the level of CHOP expression is elevated, and CHOP functions to mediate programmed cell death. Studies also found that CHOP mediates the activation of GADD34 and Ero1-Lα expression during ER stress. GADD34, in turn, dephosphorylates phospho-Ser51 of eIF2α, thereby stimulating protein synthesis. Ero1-Lα promotes oxidative stress inside the endoplasmic reticulum (ER). The role of CHOP in the programmed cell death of ER-stressed cells is correlated with its function in promoting protein synthesis and mitigating oxidative stress within the ER. CHOP protein can be detected in blood using enzyme-linked immunoassays (ELISA). This protein is not normally present in circulation and its presence reflects endoplasmic reticulum (ER) stress. Commercial ELISA kits are available for serum samples, for example the Human CHOP ELISA Kit from MyBioSource (Cat #MBS452528).

### Dosage Forms and Administration

The dosages of an agent that reduces ER stress levels to be administered can be determined by one of ordinary skill in the art depending on the clinical severity of the disorder (e.g., SD), the age and weight of the patient, the exposure of the patient to conditions that may precipitate sleep deprivation, other accompanying risk factors for ER stress, and other pharmacokinetic factors generally understood in the art, such as liver and kidney metabolism. The interrelationship of dosages for animals of various sizes and species and humans based on mg/m³ of surface area is described by E. J. Freireich et al., “Quantitative Comparison of Toxicity of Anticancer Agents in Mouse, Rat, Hamster, Dog, Monkey and Man,” Cancer Chemother. Rep. 50: 219-244 (1966). Adjustments in the dosage regimen can be made to optimize the therapeutic response. Doses can be divided and administered on a daily basis or the dose can be reduced proportionally depending on the therapeutic situation.

In some embodiments, an agent that reduces ER stress levels is administered to a subject who is sleep-deprived to treat and/or prevent damage due to SD. The dosage range depends upon the potency, and includes amounts large enough to produce the desired effect, e.g., a reduction of ER stress levels and/or a decrease of cellular damage. The dosage should not be so large as to cause unacceptable adverse side effects. Generally, the dosage will vary with the type of agent (e.g., an antibody or fragment, small molecule, siRNA, etc.), and with the age, condition, and sex of the patient The dosage can be determined by one of skill in the art and can also be adjusted by the individual physician in the event of any complication. Typically, the dosage will range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 g/kg body weight to 30 g/kg body weight. Alternatively, the dose range will be titrated to maintain serum levels between 5 g/mL and 30 g/mL.

The means by which the agent, e.g., the agent that reduces ER stress levels described herein should be administered should be appropriate for the given agent. Typically, but not necessarily, agents are administered orally. Agents can be administered, for example, in conventional pill or liquid form. If administered in pill form, they can be administered in conventional formulations with excipients, fillers, preservatives, and other typical ingredients used in pharmaceutical formations in pill form. Typically, the agents are administered in a conventional pharmaceutically acceptable formulation, typically including a pharmaceutically acceptable carrier. Conventional pharmaceutically acceptable carriers known in the art can include alcohols, e.g., ethyl alcohol, serum proteins, human serum albumin, liposomes, buffers such as phosphates, water, sterile saline or other salts, electrolytes, glycerol, hydroxymethylcellulose, propylene glycol, polyethylene glycol, polyoxyethylenesorbitan, other surface active agents, vegetable oils, and the like. A pharmaceutically-acceptable carrier within the scope of the present technology meets industry standards for sterility, isotonicity, stability, and non-pyrogenicity.

A pharmaceutically acceptable formulation can also be in pill, tablet, or lozenge form as is known in the art, and can include excipients or other ingredients for greater stability or acceptability. For the tablets, the excipients can be inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc, along with the agent that reduces ER stress levels and other ingredients.

In one embodiment, the agent that reduces ER stress levels are formulated as an enteric coated composition. In one embodiment, the agent that reduces ER stress levels are administered to the subject as an enteric coated composition. As used herein, “enteric coated composition” refers to any method that can be administered orally but is not degraded or activated until the device enters the intestines. Such methods can utilize a coating or encapsulation that is degraded using e.g., pH dependent means, permitting protection of the agent to be administered or transplanted throughout the upper gastrointestinal tract until the device reaches the alkaline pH of the intestines. In one embodiment, the enteric coated composition comprises a capsule or a pill. Such compositions are known to those of skill in the art. Material that can be used in enteric coatings includes, for example, alginic acid, cellulose acetate phthalate, plastics, waxes, shellac, and fatty acids (e.g., stearic acid, palmitic acid). Enteric coatings are described, for example, in U.S. Pat. Nos. 5,225,202, 5,733,575, 6,139,875, 6,420,473, 6,455,052, and 6,569,457, all of which are herein incorporated by reference in their entirety. The enteric coating can be an aqueous enteric coating. Examples of polymers that can be used in enteric coatings include, for example, shellac (trade name EmCoat 120 N, Marcoat 125); cellulose acetate phthalate (trade names AQUACOAT, AQUACOAT ECD, SEPIFILM™, KLUCEL™, and METOLOSE™); polyvinylacetate phthalate (trade name SURETERIC™); and methacrylic acid (trade name EUDRAGIT™). In one embodiment, the enteric-coating comprises a polymer, nanoparticle, fatty acid, shellac, or a plant fiber. In one embodiment, the enteric-coating composition is in the form of a capsule, gel, pastille, tablet or pill.

Agents that reduce ER stress levels can also be administered in liquid form in conventional formulations that can include preservatives, stabilizers, coloring, flavoring, and other generally accepted pharmaceutical ingredients. Typically, when the agents are administered in liquid form, they will be in aqueous solution. The aqueous solution can contain buffers, and can contain alcohols such as ethyl alcohol or other pharmaceutically tolerated compounds.

An agent can comprise, for example, at least 0.1%, or at least 1%, or at least 2%, or at least 3%, or at least 4%, or at least 5%, or at least 6%, or at least 7%, or at least 8%, or at least 9%, or at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90%, or at least 91%, or at least 92%, or at least 93%, or at least 94%, or at least 95%, or at least 96%, or at least 97%, or at least 98%, or at least 99% by weight of a formulation useful in the methods and compositions described herein.

A variety of means for administering an agent that reduces ER stress levels to subjects are known to those of skill in the art. Such methods can include, but are not limited to oral, parenteral, intravenous, subcutaneous, transdermal, airway (aerosol), pulmonary, cutaneous, topical, or injection. Administration can be local or systemic. When administered to treat, prevent or ameliorate an increase in ER stress levels and/or tissue damage to the gut, oral administration is preferred, although administration via the rectum, e.g., via suppository or other appropriate dosage form is contemplated.

The agent that reduces ER stress levels can be administered from once per day up to at least five times per day, depending on the severity of the SD-induced damage and anticipated duration of SD, the total dosage to be administered, and the judgment of the treating physician. In some cases, the drugs need not be administered on a daily basis, but can be administered every other day, every third day, or on other such schedules, again depending upon the anticipated duration of SD.

Therapeutic compositions containing an agent that reduces ER stress levels can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

A therapeutically effective amount is an amount of an agent that reduces ER stress levels sufficient to produce a statistically significant, measurable change in e.g., reversal of damage, etc. (see “Efficacy Measurement” below). Such effective amounts can be gauged in clinical trials as well as animal studies for a given reduction agent.

### Efficacy Measurement

The efficacy of a given treatment or prevention for damage caused by sleep deprivation can be determined by the skilled clinician. However, a treatment is considered “effective treatment,” as the term is used herein, if any one or all of the signs or symptoms of, as but one example, damage localized to a site are altered in a beneficial manner, or other clinically accepted symptoms or markers of disease are improved or ameliorated, e.g., by at least 10% following treatment with an agent that reduces ER stress levels as described herein. Efficacy can also be measured by failure of an individual to worsen as assessed by need for medical interventions (e.g., progression of damage and/or an increase of ER stress levels are halted or at least slowed). Markers for inflammatory bowel disease, a disease characterized by its cellular damage, are appropriate markers for assessing SD-induced damage. These markers include increased levels of proinflammatory cytokines, such as interleukin-1 and -8 and tumor necrosis factor, and increased calprotectin or lactoferrin. Methods of measuring these indicators are known to those of skill in the art and/or described herein. Treatment includes any treatment of a disease in an individual or an animal (some non-limiting examples include a human, or a mammal) and includes: (1) inhibiting the damage, e.g., arresting, or slowing damage induced by SD; or (2) relieving the damage, e.g., causing regression of symptoms, reducing the damage by at least 10%; and (3) preventing or reducing the likelihood of the further damage.

Assays for assessing nutrient malabsorption are known in the art and can be executed by a skilled practitioner. Primary indicators include serum triglycerides, essential fatty acids, vitamin levels, and fecal fat assays. Exemplary assays are provided herein in Table 1.

TABLE 1
Assays for Assessing Nutrient Malnutrition

| Nutrient Class | Biomarker/Analyte | Assay/Method | Example Clinical Interpretation |
|---|---|---|---|
| Protein | Serum albumin | Colorimetric assay, | Low levels suggest chronic protein |
| | | immunoturbidimetry | deficiency, liver disease, or |
| | | | inflammation |
| | Prealbumin | Immunoassay | Sensitive marker for acute changes |
| | (transthyretin) | | in protein status; low in |
| | | | malnutrition or acute illness |
| | Total nitrogen | Kjeldahl method | Estimates total body protein |
| | | | turnover |
| Carbohydrates | Fasting blood glucose | Enzymatic | Low levels may indicate poor |
| | | (hexokinase) assay | carbohydrate intake or metabolism |
| | HbA1c | HPLC or | Chronic low values may suggest |
| | | immunoassay | inadequate carbohydrate |
| | | | availability |
| Fats/Lipids | Serum triglycerides | Enzymatic assay | Low levels may indicate fat |
| | | | malabsorption or essential fatty |
| | | | acid deficiency |
| | Essential fatty acids | Gas chromatography | Deficiency linked to skin, |
| | (linoleic, α-linolenic | | immune, and growth abnormalities |
| | acids) | | |
| Fat-Soluble | Vitamin A (retinol) | HPLC | Low levels indicate poor dietary |
| Vitamins | | | intake, fat malabsorption, or liver |
| | | | dysfunction |
| | Vitamin D | Immunoassay, | Low levels suggest malabsorption |
| | (25(OH)D) | LC-MS/MS | or inadequate intake |
| | Vitamin E (α- | HPLC | Low in fat malabsorption or |
| | tocopherol) | | abetalipoproteinemia |
| | Vitamin K | Prothrombin time | Prolonged PT may indicate |
| | | (PT) | deficiency |
| Water-Soluble | Vitamin B1 (thiamine | HPLC | Low levels cause beriberi or |
| Vitamins | pyrophosphate) | | Wernicke's encephalopathy |
| | Vitamin B6 | Enzymatic or HPLC | Deficiency affects |
| | (pyridoxal-5′- | | neurotransmitter synthesis |
| | phosphate) | | |
| | Vitamin B12 | Immunoassay | Low in pernicious anemia, |
| | (cobalamin) | | malabsorption syndromes |
| | Folate | Immunoassay, | Low in malnutrition, alcoholism, |
| | | microbiological | or celiac disease |
| | | assay | |
| | Vitamin C (ascorbic | HPLC | Low in scurvy, poor intake |
| | acid) | | |
| Minerals/ | Iron (serum ferritin, | Immunoassay, | Low in iron deficiency anemia |
| Trace | transferrin saturation) | colorimetric | |
| Elements | | | |
| | Zinc | Atomic absorption | Deficiency affects immune, skin, |
| | | spectroscopy | and reproductive health |
| | Magnesium | Colorimetric assay | Low in malnutrition, GI loss |
| | Calcium | Colorimetric, ion- | Low in malabsorption, vitamin D |
| | (total/ionized) | selective electrode | deficiency |
| | Selenium | ICP-MS | Deficiency affects antioxidant |
| | | | defense and thyroid function |

An effective amount for the treatment of SD-induced damage means that amount which, when administered to a mammal in need thereof, is sufficient to result in effective treatment as that term is defined herein, for that defect. Efficacy of an agent can be determined by assessing physical indicators of SD-induced damage, such as e.g., cellular damage, as well as by evaluating the well-being and alertness of the subject receiving treatment.

The term “effective amount” as used herein refers to the amount of an agent that reduces ER stress levels as described herein needed to alleviate at least one or more symptom of SD, and relates to a sufficient amount of pharmacological composition to provide the desired effect. The term “therapeutically effective amount” therefore refers to an amount of a composition that is sufficient to provide a particular anti-damage effect when administered to a sleep deprived, typical subject. An effective amount as used herein, in various contexts, would also include an amount sufficient to delay the development of a symptom of the disorder, alter the course of a symptom (for example but not limited to, slowing the progression of a symptom of the disorder), or reverse a symptom of the disorder. Thus, it is not generally practicable to specify an exact “effective amount.” However, for any given case, an appropriate “effective amount” can be determined by one of ordinary skill in the art using only routine experimentation. The term “effective amount” is used interchangeably with the term “therapeutically effective amount” and refers to the amount of at least one agent, e.g., an agent that reduces ER stress levels, of a pharmaceutical composition, at dosages and for periods of time necessary to achieve the desired therapeutic result, for example, to reduce or stop at least one symptom of SD-induced damage, in the subject.

Effective amounts, toxicity, and therapeutic efficacy can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dosage can vary depending upon the dosage form employed and the route of administration utilized. The dose ratio between toxic and therapeutic effects is the therapeutic index and can be expressed as the ratio LD₅₀/ED₅₀. Compositions and methods that exhibit large therapeutic indices are preferred. A therapeutically effective dose can be estimated initially from in vivo assays. Also, a dose can be formulated in animal models to achieve a circulating plasma concentration range that includes the IC₅₀ (i.e., the concentration of the active ingredient, which achieves a half-maximal inhibition of symptoms). Levels in plasma can be measured, for example, by high performance liquid chromatography or other appropriate technique. It is contemplated that the relevant level for an agent that reduces ER stress levels may also be the level achieved in the lumen of the gut, as opposed to a circulating level. The effects of any particular dosage can be monitored by a suitable bioassay. The dosage can be determined by a physician and adjusted, as necessary, to suit observed effects of the treatment.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the invention is not entitled to antedate such disclosure by virtue of prior invention or for any other reason. All statements as to the date or representation as to the contents of these documents is based on the information available to the applicants and does not constitute any admission as to the correctness of the dates or contents of these documents.

Some embodiments of the technology described herein can be defined according to any of the following numbered paragraphs:
1. A method for treating damage induced by sleep deprivation (SD), the method comprising: administering to an individual who is sleep deprived an agent that reduces endoplasmic reticulum (ER) stress.
2. The method of paragraph 1, wherein the damage is nutrient malabsorption.
3. The method of any preceding paragraph, wherein the damage occurs at a site selected from the group consisting of brain, gastrointestinal tract, mouth, throat, lungs, heart, liver, gut, stomach, kidney, skin, bones, large intestine, small intestine, bladder, and muscular system.
4. The method of any preceding paragraph, wherein the damage occurs in the gut.
5. The method of any preceding paragraph, wherein SD is chronic or acute.
6. The method of any preceding paragraph, wherein the agent is selected from the group consisting of a compound, a small molecule, a food additive, and an enzyme.
7. The method of any preceding paragraph, wherein the small molecule is 4-Phenylbutyric acid or Tauroursodeoxycholic acid.
8. The method of any preceding paragraph, further comprising the step of, prior to administering, diagnosing the individual of having or being at risk of having damage induced by SD.
9. The method of any preceding paragraph, further comprising the step of, prior to administering, receiving results of an assay that diagnosing the individual as having or being at risk of having damage induced by SD.
10. A composition for treating or preventing damage induced by SD, the composition comprising an agent that reduces ER stress and a sedative.
11. The composition of any preceding paragraph, wherein the agent is a small molecule selected from 4-Phenylbutyric acid or Tauroursodeoxycholic acid.
12. The composition of any preceding paragraph, wherein the sedative is selected from a group consisting of: a barbiturate, a benzodiazepine, a non-benzodiazepine hypnotic, a methoaqualone, a first generation antihistamine, an antidepressant, an antipsychotics, an herbal sedative, alcohol, an opioid, a general anesthetic, a melatonin agonist, a orexin antagonists, and a skeletal muscle relaxant.
13. The composition of any preceding paragraph, wherein the composition further comprises a pharmaceutically acceptable carrier.
14. A composition for treating or preventing damage induced by SD, the composition comprising: an agent that reduces ER stress and a stimulant.
15. The composition of any preceding paragraph, wherein the agent is a small molecule selected from 4-Phenylbutyric acid or Tauroursodeoxycholic acid.
16. The composition of any preceding paragraph, wherein the stimulant is selected from a group consisting of: an herbal stimulant, an amphetamine, a methamphetamine, cocaine, a methylxanthine, ephedrine, a cathinone, mephedrone, methylenedioxypyrovalerone, methylenedioxymethamphetamine, nicotine, propylhexedrine, and pseudoephedrine.
17. The composition of any preceding paragraph, wherein the composition further comprises a pharmaceutically acceptable carrier.
18. A method for treating or preventing damage induced by SD, the method comprising: administering to an individual who is sleep deprived a composition of any preceding paragraph.

### EXAMPLES

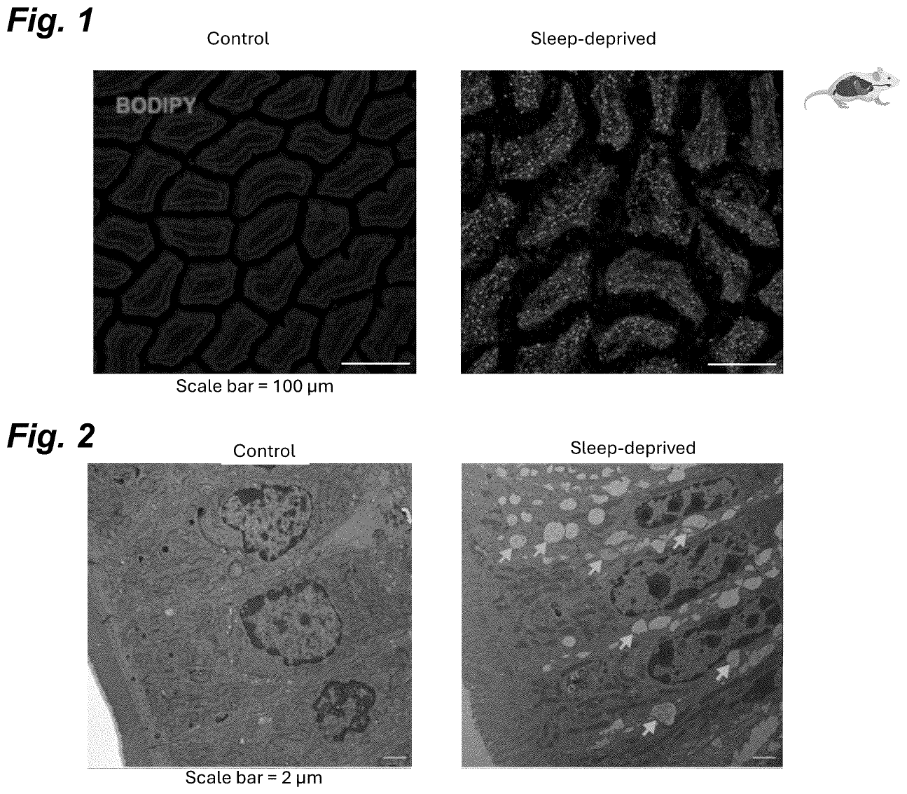

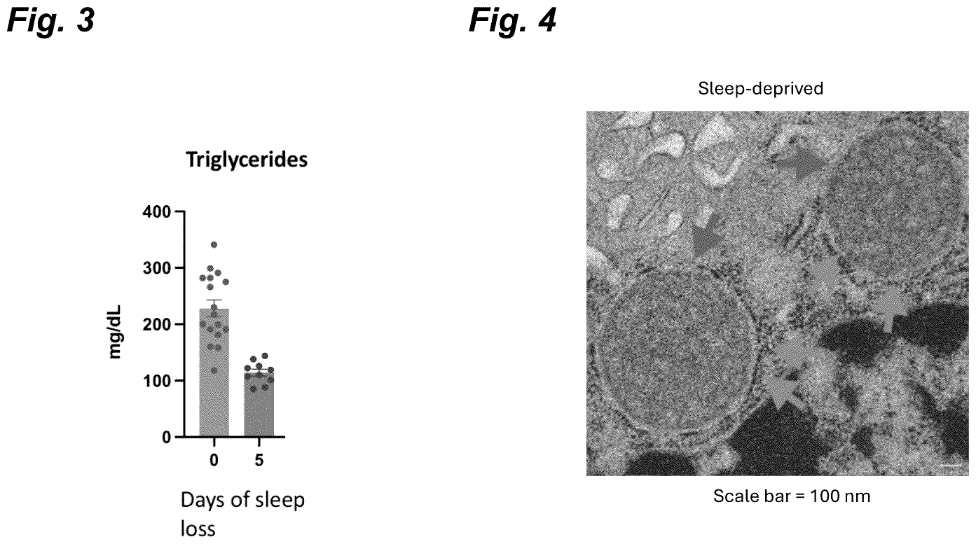

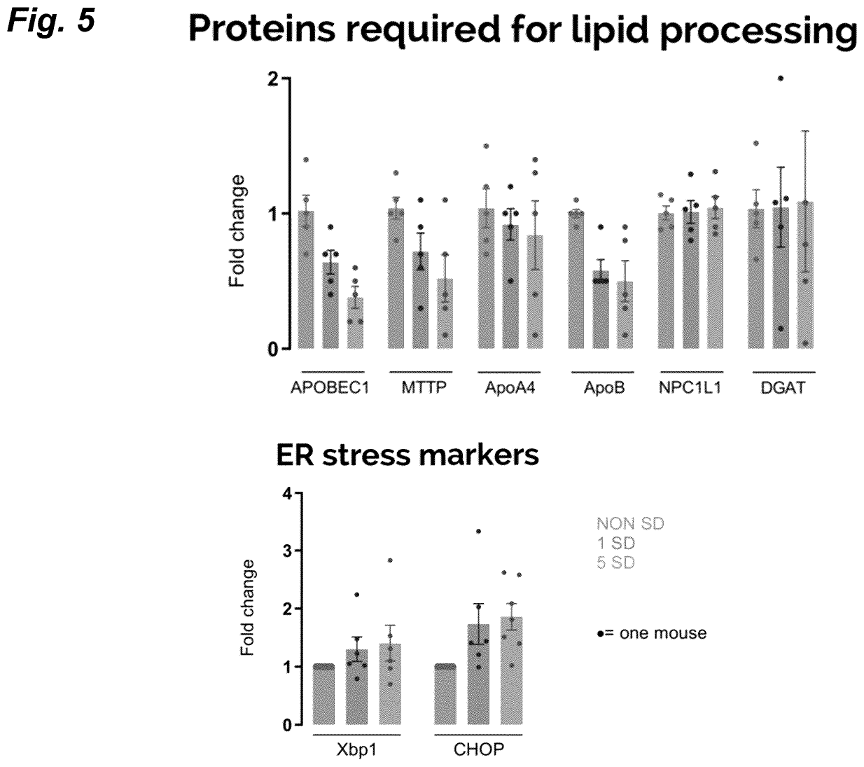

The invention is based in part on the discovery that sleep loss causes nutrient malabsorption in the intestine. Data presented herein show that SD leads to an increase of ER stress, causing nutrient malabsorption. FIGS. 1A-2 show that lipids accumulate inside the enterocytes of mice which have been sleep deprived. As a consequence of lipid accumulation in the enterocytes, not enough lipids are absorbed into the circulation. FIG. 3 shows that the triglyceride levels are low in the circulation of sleep-deprived mice.

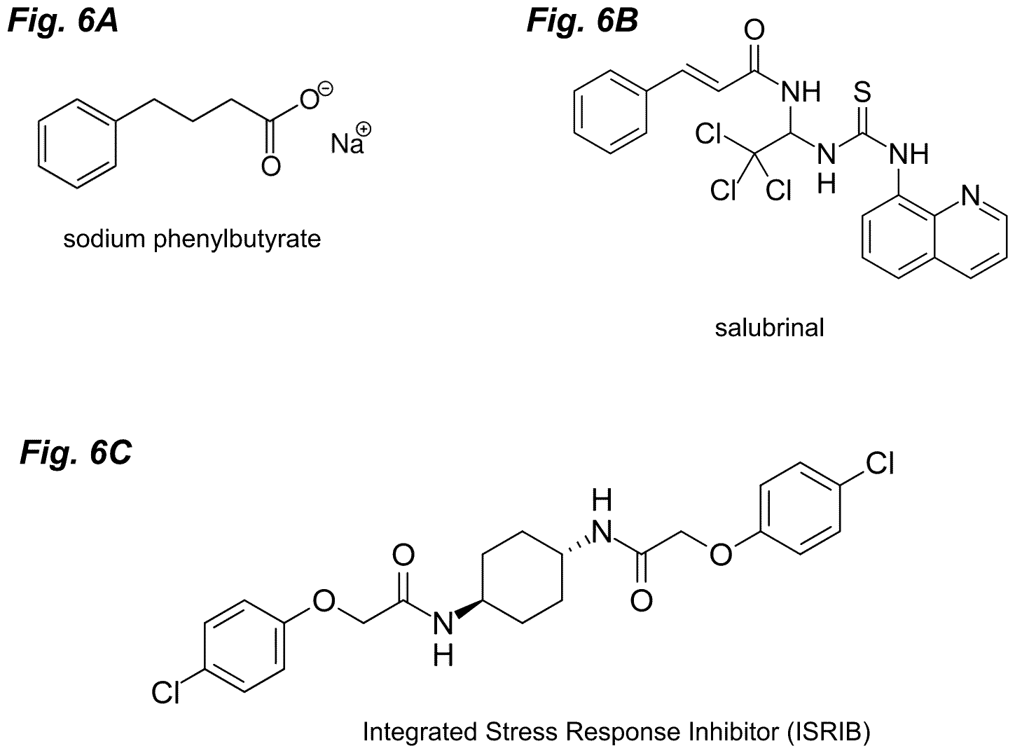

The problems with absorption start after one day of sleep restriction, which is before any ROS are detectable (the earliest ROS detection is after 48 hours of sleep restriction). Also after only one day of sleep loss problems are detected in the Endoplasmic Reticulum (ER). FIG. 4 shows the intestines of mice which were sleep-deprived for 24 hours (˜50% sleep loss).

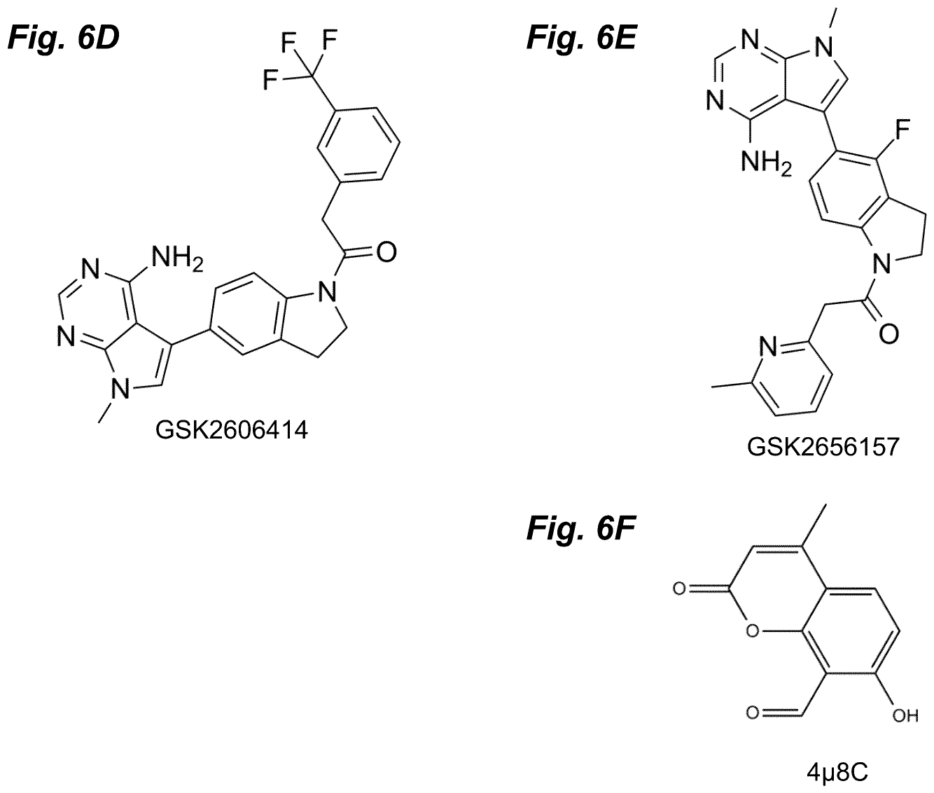

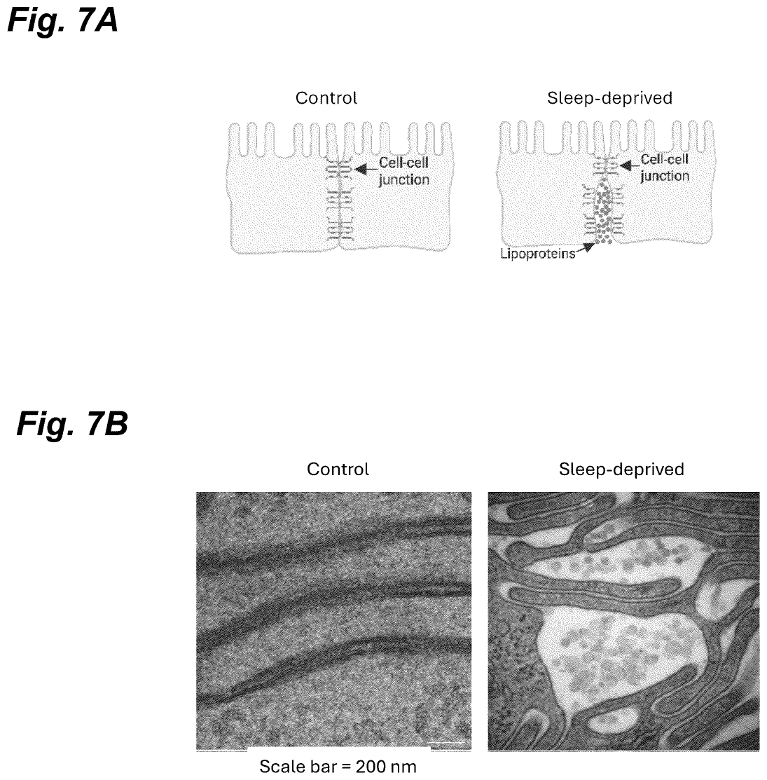

Lipids need to be processed through the ER before absorption. FIG. 5 shows reduced levels of several proteins which are needed for this processing, as well as elevated levels of ER stress markers, in sleep-restricted mice. FIG. 7 shows at the level of electron microscopy that tight junctions between enterocytes in the gut are disrupted in sleep-deprived mice. In contrast to what is observed in non-sleep-deprived animals, the spaces between cells in the sleep-deprived animals are filled with lipids and with particles that appear to be chylomicrons.

It is specifically contemplated herein that lipids accumulate in the intestines of sleep-restricted animals due to ER stress. Accordingly, preventing or treating ER stress will prevent and/or treat (i.e., lessen) lipid accumulation in the intestine. Exemplary ER stress inhibitors for the prevention or treatment of ER stress include, but are not limited to, 4-Phenylbutyric acid and Tauroursodeoxycholic acid.

What is claimed is:

1) A method for treating damage induced by sleep deprivation (SD), the method comprising: administering to an individual who is sleep deprived an agent that reduces endoplasmic reticulum (ER) stress.

2) The method of claim 1, wherein the damage is nutrient malabsorption.

3) The method of claim 1, wherein the damage occurs at a site selected from the group consisting of: brain, gastrointestinal tract, mouth, throat, lungs, heart, liver, gut, stomach, kidney, skin, bones, large intestine, small intestine, bladder, and muscular system.

4) The method of claim 1, wherein the damage occurs in the gut.

5) The method of claim 1, wherein SD is chronic or acute.

6) The method of claim 1, wherein the agent is selected from the group consisting of a compound, a small molecule, a food additive, and an enzyme.

7) The method of claim 6, wherein the small molecule is 4-Phenylbutyric acid or Tauroursodeoxycholic acid.

8) The method of claim 1, further comprising the step of, prior to administering, diagnosing the individual of having or being at risk of having damage induced by SD.

9) The method of claim 1, further comprising the step of, prior to administering, receiving results of an assay that diagnosing the individual as having or being at risk of having damage induced by SD.

10) A composition for treating or preventing damage induced by SD, the composition comprising an agent that reduces ER stress and a sedative.

11) The composition of claim 10, wherein the agent is a small molecule selected from 4-Phenylbutyric acid or Tauroursodeoxycholic acid.

12) The composition of claim 10, wherein the sedative is selected from a group consisting of a barbiturate, a benzodiazepine, a non-benzodiazepine hypnotic, a methoaqualone, a first generation antihistamine, an antidepressant, an antipsychotics, an herbal sedative, alcohol, an opioid, a general anesthetic, a melatonin agonist, a orexin antagonists, and a skeletal muscle relaxant.

13) The composition of claim 10, wherein the composition further comprises a pharmaceutically acceptable carrier.

14) A composition for treating or preventing damage induced by SD, the composition comprising: an agent that reduces ER stress and a stimulant.

15) The composition of claim 14, wherein the agent is a small molecule selected from 4-Phenylbutyric acid or Tauroursodeoxycholic acid.

16) The composition of claim 14, wherein the stimulant is selected from a group consisting of an herbal stimulant, an amphetamine, a methamphetamine, cocaine, a methylxanthine, ephedrine, a cathinone, mephedrone, methylenedioxypyrovalerone, methylenedioxymethamphetamine, nicotine, propylhexedrine, and pseudoephedrine.

17) The composition of claim 14, wherein the composition further comprises a pharmaceutically acceptable carrier.

18) A method for treating or preventing damage induced by SD, the method comprising: administering to an individual who is sleep deprived a composition of claim 10.

19) A method for treating or preventing damage induced by SD, the method comprising: administering to an individual who is sleep deprived a composition of claim 14.
